(19) United States
(12) Patent Application Publication
(10) Pub. No.: US 2024/0218460 A1
(43) Pub. Date: Jul. 4, 2024

(54) COMPOSITIONS AND METHODS OF ENHANCING PHOTOSYNTHESIS

(71) Applicant: Miami University, Oxford, OH (US)

(72) Inventors: Xin Wang, Gainesville, FL (US); Zhenxiong Jiang, Gainesville, FL (US)

(21) Appl. No.: 18/400,586

(22) Filed: Dec. 29, 2023

Related U.S. Application Data
(60) Provisional application No. 63/436,245, filed on Dec. 30, 2022.

(51) Int. Cl. C12Q 1/6895; C12N 1/20; C12N 15/82; C12R 1/01

(52) U.S. Cl. CPC C12Q 1/6895; C12N 1/205; C12N 15/8245; C12R 2001/01

(57) ABSTRACT

Disclosed herein are compositions, systems, and methods for directed adaptive enhancement of host cells to enhance photosynthesis. In certain embodiments, the methods include genetic modification of an organism (e.g., a cyanobacterium) to increase mutagenic frequency and providing one or more stressors to the modified cell to induce an adaptive response, screening the resulting population to identify candidates with a desired phenotype (genetic outcome) e.g., increased growth or production of one or more desirable biological products. In certain embodiments, the disclosure provides a modified bacteria with enhanced sucrose production and/or growth and methods of producing a modified bacteria characterized by enhanced sucrose production and/or growth relative to wild-type.

### CROSS-REFERENCE TO RELATED APPLICATIONS

This non-provisional application claims priority to and the benefit of U.S. Provisional application No. 63/436,245, filed on Dec. 30, 2022, the content of which is hereby incorporated by reference in its entirety.

### FIELD

The present disclosure relates to compositions for and methods of enhancing a rate of adaptive mutation, more particularly to achieving increases in photosynthesis and growth of cyanobacterial species, including enhancing growth or the production of one or more biological products.

### BACKGROUND

Estimates indicate that global food production will not meet demand of the growing population in the coming decades. Efforts are in place to achieve higher global output, including genetic modification. Modification of bacterial species to optimize one or more products of the bacteria is a well-known field of study. Bacteria have been modified for e.g., biofuel production, selective waste degradation (e.g., to reduce landfill mass or fossil fuel spill cleanup). These modifications have been performed thru methods such as selective breeding and targeted genetic modification.

Cyanobacteria have been well studied as organisms that utilize energy from light for various metabolic processes and the production of biomass due to the diversity of the species and their optimal environments in which they thrive. These processes and a number of other characteristics such as versatile metabolism, morphological changes, and tolerance to abiotic stresses have led to a diverse group of microorganisms due to adaptation strategies.

Synechococcus elongatus PCC 7942 is a well-studied organism in which a number of molecular techniques have been developed and cellular pathways have been mapped. The ability of this organism to adapt to its ever-changing environment has been a mechanism of interest for some time now and discoveries such as synthesis of compatible solutes, regulation of membrane transporters, and alteration of cellular physiology and structure have allowed a greater understanding of these organisms.

### SUMMARY

The general inventive concepts are based, in part, on the discovery that directed, adaptive (hyper)evolution can be induced in a predetermined fashion in certain host cells (e.g., cyanobacterial species) to achieve one or more desirable adaptive mutants. Desirable adaptations include host cells displaying one or more of enhanced growth (e.g., biomass accumulation) and/or enhanced production of one or more desirable biological products (e.g., sucrose).

Disclosed herein are compositions, systems, and methods for directed and accelerated mutation of biological cells. In certain aspects, the general inventive concepts contemplate a method of identifying a genetic mutation capable of enhancing at least one of photosynthetic rate and efficiency, the method comprises modification of a plurality of bacterial cells to enhance mutation rate relative to wild-type organisms; culturing the modified bacterial cells in the presence of a stressor; identifying modified cells that demonstrate an enhanced marker of photosynthetic rate and/or efficiency; and identifying the genetic modification that leads to the enhanced photosynthetic rate and/or efficiency.

In certain exemplary embodiments, the general inventive concepts contemplate a method of achieving greater photosynthetic rate and/or efficiency in a bacterial species. The method comprises modification of a plurality of bacterial cells to enhance mutation rate relative to wild-type organisms; culturing the modified bacterial cells in the presence of a stressor; identifying modified cells that demonstrate an enhanced marker of photosynthetic rate and/or efficiency; and identifying the phenotype of interest that leads to the enhanced photosynthetic rate and/or efficiency.

In certain exemplary embodiments, the methods include genetic modification of an organism to increase mutagenic frequency (or mutation rate) and providing one or more stressors to the modified cell to induce a predetermined genetic outcome e.g., increased growth and/or production of one or more desirable biological products (e.g., level of sucrose). In certain embodiments, the disclosure provides a modified bacteria with enhanced sucrose production and methods of producing modified cyanobacteria capable of enhanced sucrose production relative to wild-type. In certain embodiments, the general inventive concepts contemplate an iterative method using high through-put screening technology to identify appropriate cells or strains based on modeling. In certain embodiments, the disclosure provides a modified bacteria with enhanced growth/growth rate and methods of producing modified cyanobacteria capable of enhanced growth rate relative to wild-type. In particular, Applicants have demonstrated that the ability of e.g., Synechococcus elongatus PCC 7942, to adapt to a stressor (e.g., high salt concentrations) introduced in the environment can be used to drive adaptive evolutionary changes providing strains with desirable traits.

In certain exemplary embodiments, the general inventive concepts contemplate compositions, systems, and methods for enhancing the growth of cyanobacteria, including enhancing photosynthetic rate. Markers include sucrose production and growth, among others.

In certain exemplary embodiments, the general inventive concepts contemplate compositions, systems, and methods for enhancing production of one or more desirable biological products of cyanobacteria, including but not limited to sucrose.

In certain exemplary embodiments, the general inventive concepts contemplate a method of enhancing production of one or more desirable biological products, the method comprising contacting a host cell with a vector capable of inserting a nirA promoter in front of a mismatch repair pathway gene to form a modified host cell, providing predetermined levels of nitrogen to the modified host cell, culturing the modified host cell in the presence of a stressor, measuring the level of the one or more desirable biological products, and identifying one or more modified host cell strains that demonstrate enhanced production of the desirable biological product. In certain embodiments, the general inventive concepts contemplate an iterative method using high through-put screening technology to identify appropriate cells or strains.

In certain exemplary embodiments, the general inventive concepts contemplate a method of stimulating adaptive mutation in a host cell to produce phenotypic changes of interest, the method comprises modifying of a plurality of cyanobacterial cells to form modified cyanobacterial cells; culturing the modified cyanobacterial cells in the presence of an environmental stressor; identifying modified cells that demonstrate at least one phenotypic change of interest; and further culturing the identified cells in the presence of an environmental stressor.

Applicants have demonstrated that modification of a bacterium (e.g., cyanobacteria) via insertion of a nirA promoter into the genome of a cyanobacterium (i.e., Synechococcus elongatus PCC 7942) in front of the main mismatch repair pathway gene involved in recognizing mismatch bases, mutS, can increase the rate of adaptive evolution/mutation in the presence of a stressor. By inserting this promoter Applicants were able to control the expression of mutS on a transcriptional level allowing directed but spontaneous mutations to occur. In certain embodiments, the mutations are initiated through modification of nitrogen sources in the media.

Other aspects and features of the general inventive concepts will become more readily apparent to those of ordinary skill in the art upon review of the following description of various exemplary embodiments in conjunction with the accompanying figures.

### BRIEF DESCRIPTION OF THE DRAWINGS

The general inventive concepts, as well as embodiments and advantages thereof, are described below in greater detail, by way of example, with reference to the drawings in which:

FIG. 1 is an image of RT-PCR analysis of the relative expression of mutS compared to housekeeping gene rpnB in both the wild-type and the mutS0:: nirAp (Km^{R}) cells incubated in BG11 medium containing either nitrate or ammonium as the main nitrogen source.

FIG. 2 shows a spot assay analysis to examine growth of mutS0:: nirAp (Km^{R}) cells on BG11 agar plates with either nitrate or ammonium as the main nitrogen source.

FIG. 3 is a graph showing primary sucrose screen of Se1 mutants following 18 hours of induction with 125 mM NaCl. Intracellular sucrose was examined in WT, mutS0:: nirAp (Km^{R}), and Se1 strains. The strain expressing the sucrose permease, CscB (MSU), was induced with 1 mM IPTG in addition to the NaCl for extracellular sucrose to be detected and used as the control to compare with Se1 mutant strains.

FIG. 4A is a plot showing the validation assay for strains that exhibited a 2-fold or greater increase in sucrose productivities in the primary screening after an 18-hour induction in 150 mM NaCl. This graph is confirmation that Se1 strains Se1-728 and Se1-1086 as potential elite sucrose producers.

FIG. 4B is a plot showing the validation assay for strains that exhibited a 3-fold or greater increase in sucrose productivities in the primary screening after an 18-hour induction in 150 mM NaCl. This graph is further confirmation that Se1 strains Se1-728 and Se1-1086 as potential elite sucrose producers.

FIG. 5 is a bar graph showing in 96 well plates the increased extracellular sucrose productivity in Se1-1086 by the overexpression of cscB after 18 hours of 150 mM NaCl and 1 mM IPTG induction. Error bars indicate the SD of three biological replicates. Statistical significance was determined by a one-tailed Student's t-test. *P≤0.05, **P≤0.01.

FIG. 6 is a bar graph showing a scale-up analysis of Se1-1086 CscB and CscB (MSU) with increased extracellular sucrose productivity in Se1-1086 CscB after 48 hours in 150 mM NaCl and 1 mM IPTG. Error bars indicate SD of three biological replicates. Statistical significance determined through a one-tailed Student's t-test. P≥0.05.

FIG. 7 is a graph showing growth curve analysis in a 24 well plate. Optical densities were read at 730 nm in the Molecular Devices SpectraMax iD5 series microplate reader. Cells were grown in BG11 (NO₃⁻) medium without salt for 4 days and on day 4 they were induced with 125 mM NaCl. This graph shows the improvement of growth in several Se1 strains (Se1-314, 316, 340) compared to the control strain mutS0:: nirAp (Km^{R}).

FIG. 8 is a graph showing sucrose screening of Se2 strains after 30 hours in 150 mM NaCl compared to both mutS0:: nirAp and Se1-1086 strains. Each panel shows an independent intracellular sucrose assay of different Se2 strains with controls. Mean values of technical replicates are shown in each panel.

FIG. 9 is a graph showing confirmation analysis of higher sucrose producing Se2 strains show potential elite sucrose producers at 150 mM NaCl after 30 hours. Each panel shows the mean values of separate individual sucrose assays using the same strains each assay was ran with four biological replicates.

FIG. 10 is a graph showing scale up analysis of Se2 strains engineered with sucrose transporters after 24 hours in 150 mM NaCl and 1 mM IPTG. Mean values for two biological replicates and two technical replicates shown with error bars indicative of SD. Statistical analysis performed using a one-tailed student's t-test. *P≤0.05, **P≤0.01, ***P≤0.001

FIG. 11 is a graph showing a third round of high throughput screening of about 10,000 cyanobacterial mutants (mSe3) following 30 hours of induction of 150 mM NaCl. Both growth (OD730 nm) and sucrose production were monitored. Panel A shows the screening of the ˜10,000 mSe3 mutants (gray dots) with the top 1% of sucrose-producers highlighted in green and the top 0.5% of mutants with high biomass accumulation highlighted in blue. The eight strains in boxes are the ones confirmed through validation experiment. Panel B shows a subset of the 10,000 Se3 mutants analyzed by the biomass fold change. Two strains mSe3-7265 and mSe3-8345 were found to be the fast-growing mutants. Panel C and D have the same data as A and B with the sucrose production represented with specific productivity rather than productivity.

FIG. 12 is a graph showing growth versus time, demonstrating a quasi-exponential growth pattern in the high through-put screening system, demonstrating the feasibility of using fold change as the screening criteria.

FIG. 13 is an image showing a sample 96-well plate showing mutants with or without chlorosis.

FIG. 14 is a graph showing the non-linear boundary of mutants with chlorosis (Blue) or not (Red) in the high throughput screening system.

FIG. 15A is a graph showing validation data demonstrating the elevated photosynthesis capacity (biomass change) of certain mutants.

FIG. 15B is a graph showing validation data demonstrating the elevated sucrose productivity of certain mutants.

FIG. 16 is a set of four graphs showing the growth dynamics of BAM mutants in the multicultivator.

FIG. 17 is an image showing the filamentous mutant mSe3-342 as an example of an interesting phenotype.

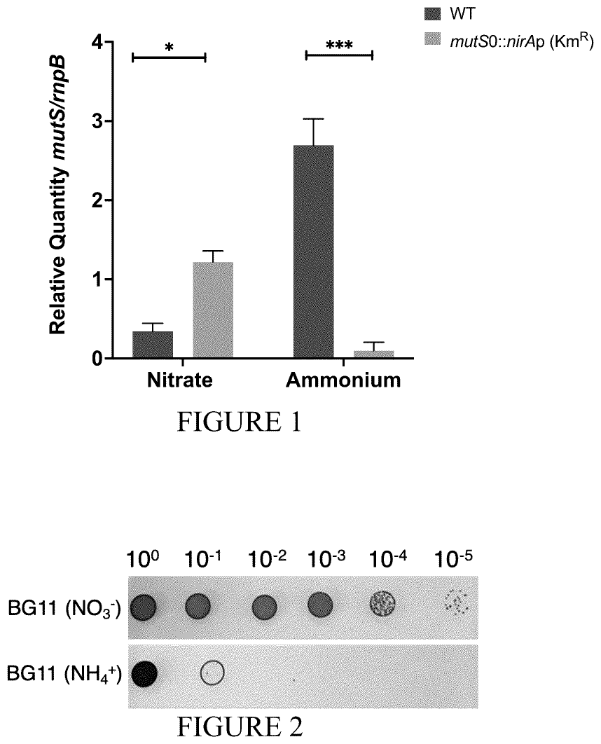

### DETAILED DESCRIPTION

Several illustrative embodiments will be described in detail with the understanding that the present disclosure merely exemplifies the general inventive concepts. Embodiments encompassing the general inventive concepts may take various forms and the general inventive concepts are not intended to be limited to the specific embodiments described herein.

While various exemplary embodiments are described or suggested herein, other exemplary embodiments utilizing a variety of methods and materials similar or equivalent to those described or suggested herein are encompassed by the general inventive concepts.

Unless defined otherwise, all technical and scientific terms used herein have the same meaning as commonly understood by one of ordinary skill in the art to which the invention belongs.

The term “vector” refers to a nucleic acid capable of transporting another nucleic acid to which it has been linked. One type of vector that can be used in accordance with the presently disclosed subject matter is a retroviral vector, i.e., a nucleic acid capable of integrating the nucleic acid sequence of interest into the host cell chromosome. Other vectors include those capable of autonomous replication and expression of nucleic acids to which they are linked. Vectors capable of directing the expression of genes to which they are operatively linked are referred to herein as “expression vectors.” In general, expression vectors of utility in recombinant DNA techniques are often in the form of plasmids. However, the presently disclosed subject matter is intended to include such other forms of expression vectors which serve equivalent functions (e.g., retroviral vectors such as adenoviral vectors, cosmids or bacmids) or and which become known in the art subsequently hereto. Vectors as described herein include one or more selective marker genes. Stable modification of a host cell with a vector as described herein provides a transformed or transfected cell (also referred to as a modified cell). Any known method may be used to integrate the nucleic acid molecule into the genome of the host cell so long as it is compatible with the general inventive concepts herein.

As used herein, the term “selective marker gene” refers to a gene whose coded product enables the transformed or transfected host cells to exhibit new or enhanced characteristics under selective pressure (e.g., marker of photosynthetic rate and/or efficiency). Such selective pressure (stressor) includes, but is not limited to, the addition of environmental stressors (e.g., solute concentration) or the lack of nutrients.

The term “modified host cell” refers to a host cell that has been modified by a vector to produce a coded product by increasing the rate of mutation from one or more stressors. In certain embodiments, the term refers to bacterial cell or strain as modified herein to accelerate the rate of mutation. In certain embodiments, the term can also refer to downstream cells or strains that have undergone more than one round of stressor exposure that leads to a change in behavior of the cell (also referred to as environment-driven genetic modification).

The term “stressor” or “environmental stressor” refer(s) to predetermined variables in the environment of the modified host cells or cells that stimulate adaptive mutations. Non-limiting examples of environmental stressors include solute concentration and available nitrogen/nutrients. Examples of suitable stressor solutes include: NaCl, KCl, and CaCl₂).

The term “accelerated mutation” or “accelerated mutation rate” refer(s) to an increase in the rate of environment driven adaptive modification (i.e., mutations) relative to a wild-type organism or otherwise unmodified organism. The term includes an increase of 10 over the expected or measured rate of genetic mutation, including 20%, 30%, 40%, 50%, 60%, 70%, 80%, 90%, 100%, 200%, 300%, 400%, 500%, 600%, 700%, 800%, 900%, and including increases of 1000% or more relative to the wild-type rate.

The term “adaptive evolution” or “adaptive mutation” refer(s) to an environment-driven genetic modification that is directed toward a desired result (e.g., a desirable phenotype including increased growth, production, or growth rate). Such adaptations are characterized or demonstrated by a marker of photosynthetic rate and/or efficiency, including enhanced photosynthetic rate and/or efficiency. Adaptive mutations can also be expressed as phenotypic changes of interest.

The term “marker of increased photosynthetic rate and/or efficiency” refer(s) to a measured value or product determined or produced during culturing a modified cell with one or more stressors. In certain instances, the marker may be a biological product, including but not limited to sucrose. In certain instances, the marker may be growth of the cell, including e.g., measured size such as diameter, volume, weight, and/or length of a cell. Mutants that demonstrate such markers of increased/enhanced photosynthetic rate and/or efficiency are referred to herein as sucrose-producing mutants (SPMs), biomass-accumulating mutants (BAMs), and fast-growing mutants (FGMs), etc.

The term “enhanced photosynthetic rate and/or efficiency” refer(s) to a measure of the photosynthesis of a modified organism relative to the wild-type or otherwise unmodified organism. The term includes an increase of 10% over the expected or measured rate of photosynthesis or photosynthetic efficiency, including 20%, 30%, 40%, 50%, 60%, 70%, 80%, 90%, and including increases of 100% or more relative to the wild-type rate.

Synechococcus elongatus PCC 7942 is one of the most well-described and studied freshwater cyanobacterium in the field due to its natural competence allowing it to be genetically malleable and its ability to populate diverse environments, including those of high salinity. This capability to survive in high salinity environments is important because salt concentrations are considered an important abiotic factor in natural habitats of microorganisms and fluctuations can indicate various changes in that environment due to both geographical factors and climate change. Salt is a large stressor to living cells because of its high ionic strength, which can lead to an influx of inorganic ions that are often toxic to cellular metabolism, and its high osmotic pressure, which reduces cell turgor and inhibits growth through direct loss of intracellular water. Adaptations to high salinity environments have been shown to evolutionarily promote cell survival and there are currently two main mechanisms that have been defined.

The first is mainly seen in halophilic Archaea and some halophilic bacteria and works by the accumulation of inorganic ions and the evolution of macromolecules that have a capacity to bind salts and water making it so the cell is able to tolerate high internal concentrations of inorganic ions.

The second mechanism of salt tolerance is the protection of intracellular macromolecules through the simultaneous pumping of ions, like Na⁺ and Cl⁻ions, out of the cytoplasmic space while accumulating compatible solutes. This allows the cellular osmotic potential to be adjusted through water uptake while also protecting the cells' integrity and their internal cellular components. The concept of compatible solutes was established in 1976 and the importance of the accumulation of compatible solutes as a method of salt tolerance in cyanobacteria is well described because of the diversity of solutes that are produced and how they can be used for further product development in the biotechnological field. Compatible solutes are often defined as small organic compounds with no net charge which give them the ability to equilibrate osmotic conditions without disturbing cellular metabolism, even at high concentrations Some of the most commonly produced compatible solutes by cyanobacteria include trehalose, glucosyl glycerol, glycine betaine, and sucrose which vary among the different species.

S. elongatus 7942 mainly produces sucrose as its compatible solute in response to high salt concentrations and the production of sucrose has been of increasing interest in the biotechnology field for a while because of it being a carbohydrate feedstock to a number of bio-industrial compounds like biofuels, nutritional supplements, and pharmaceutical components (evolution uses stress induction of a hypermutator strain to produce mutations in the organism's genome which can be screened for various applications). This allows the environment to push natural adaptations through selective pressures in either short or long-term experiments leading to altered products as key identifiers of mutation. Those of skill in the art will recognize that there is a range necessary for concentration to drive adaptive mutation that must be high enough to provide stress, while avoiding amounts that are too toxic—potentially leading to cell death. Suitable solute concentrations according to the general inventive concepts range from about 100 mM to about 500 mM, including 125 mM to 450 mM, including 150 mM to 400 mM, including 175 mM to 300 mM, including 200 mM to about 275 mM, and including about 250 mM.

Many types of conditions have been well described as mutators and as long as the chosen environment provides pressure, adaptation will occur. This method has been around in the microbial genetics community and was used in the discovery of citrate utilization by Escherichia coli. It allows for a number of long-term and short-term experiments to be done, in which both have a number of protocols that have been well defined. The utilization of these methods for increased production of certain products of interest comes from the notion of eco-evolutionary feedback. This suggests that we can trace a phenotype to an evolutionary change and vice versa. Feedbacks have been identified to generate a spatial variation by impacting population regulation, community dynamics, and promotion of coexistence between a species that has branched and now has a different phenotype. These concepts of eco-evolutionary feedback can give an idea into the evolution of a single species and the co-culturing of multiple evolved species to get a phenotypic output of interest.

Adaptive evolution is fueled by de novo mutations and the rate of evolution is proportional to the genetic variation of a population thus, there is an unlimited number of possibilities to use a combination of environmental pressures and genetic manipulations, at the same time. Adaptations may be difficult, if not impossible to predict but, with the economic feasibility, time input of each mutation round, and the ability to screen for phenotypic changes of interest, experimental evolution is a good way to drive phenotypes of interest, like sucrose production in cyanobacteria. Accordingly, the general inventive concepts are based, in part on the discovery that through adaptive evolution, sucrose production in cyanobacteria can be enhanced, including increasing sucrose production by at least 10%, at least 20%, at least 30%, at least 40%, at least 50%, at least 75%, and at least 100% relative to wild-type sucrose production.

With previous biotechnological and genetic manipulation methods, there have been very limited changes in the production of sucrose in cyanobacteria. Mainly genetic manipulations to either overexpress the main enzymes involved in sucrose synthesis have been done on sucrose phosphate synthase (SPS) and sucrose phosphate phosphatase or the redirection of carbon toward sucrose synthesis through limiting or completely knocking out the expression of ADP-glucose pyrophosphorylase (AGPasc) and glycogen synthase (GS) leading to glycogen not being produced and stored thus freeing up more carbon for sucrose synthesis. These methods however have shown very limited increases of sucrose synthesis with an eventual plateau of sucrose production capabilities in the organism most likely due to cellular regulation mechanisms. Therefore, Applicants created a hypermutator strain by inserting a nirA promoter into the genome of S. elongatus PCC 7942 in front of the main mismatch repair pathway gene involved in recognizing mismatch bases, mutS. By inserting this promoter Applicants were able to control the expression of mutS on a transcriptional level allowing spontaneous mutations to be produced and conserved through only the change of nitrogen sources present in the media. This new method allows the cell to generate random mutations and through the use of high throughput screening of sucrose production level Applicants identified enhanced sucrose producing strains.

The general inventive concepts are based, in part on the discovery that cells can be induced into adaptive evolutionary mutations by inserting a nirA promoter to regulate the expression of mutS and delivering predetermined levels of nitrogen (i.e., an environmental stressor) to the modified organisms/host cells. These mutations can thereby be directed toward enhanced production of one or more biological products, including enhanced sucrose production and/or increases in growth. Thus, in certain exemplary embodiments, the general inventive concepts include a vector for inserting a nirA promoter in front of mutS in a bacterium. In certain exemplary embodiments, the general inventive concepts include a modified host cell comprising a nirA promoter in front of mutS. In certain exemplary embodiments, the general inventive concepts contemplate a modified bacteria demonstrating decreased expression of mutS relative to wild-type.

While not wishing to be bound by theory, Applicants believe that by genetically modifying cells to increase susceptibility to mutations (e.g., by inserting a nirA promoter into the genome of a cyanobacteria in front of the main mismatch repair pathway protein involved in recognizing mismatch bases, mutS) the rate of mutations can be enhanced (increased) relative to wild-type. By increasing the rate of mutations, it is possible to provide environmental stressors to induce and direct mutations, allowing for identification of desirable strains (e.g., those the exhibit one or more of increased growth or increased production of a desirable biological product). The cells modified according to the general inventive concepts function as enhanced producers of desirable biological products (e.g., sucrose).

Thus, in certain exemplary embodiments, the general inventive concepts contemplate compositions, systems, and methods for directed and accelerated mutation of biological cells. In general, the method comprising contacting a host cell (e.g., a bacteria cell) with a vector capable of inserting a nirA promoter in front of a mismatch repair pathway protein to form a modified host cell, providing predetermined levels of nitrogen to the modified host cell, culturing the modified host cell in the presence of a stressor, identifying one or more modified host cell strains that demonstrate enhanced production or growth of the desirable biological product, and identifying the genetic modification that leads to the enhanced production or growth.

Disclosed herein are compositions, systems, and methods for directed and accelerated mutation of biological cells. In certain aspects, the general inventive concepts contemplate a method of identifying a genetic mutation capable of enhancing at least one of photosynthetic rate and efficiency, the method comprises modification of a plurality of bacterial cells to enhance mutation rate relative to wild-type organisms; culturing the modified bacterial cells in the presence of a stressor; identifying modified cells that demonstrate an enhanced marker of photosynthetic rate and/or efficiency; and identifying the genetic modification that leads to the enhanced photosynthetic rate and/or efficiency.

In certain aspects, the general inventive concepts contemplate a method of achieving greater photosynthetic rate and/or efficiency in a bacterial species. The method comprises modification of a plurality of bacterial cells to enhance mutation rate relative to wild-type organisms; culturing the modified bacterial cells; identifying modified cells that demonstrate an enhanced marker of photosynthetic rate and/or efficiency; and identifying the genetic modification that leads to the enhanced photosynthetic rate and/or efficiency.

In certain embodiments, the methods include genetic modification of an organism to increase mutagenic frequency (or mutation rate) and providing one or more stressors to the modified cell to induce a predetermined genetic outcome e.g., increased growth and/or production of one or more desirable biological products (e.g., level of sucrose). In certain embodiments, the disclosure provides a modified bacteria with enhanced sucrose production and methods of producing modified cyanobacteria capable of enhanced sucrose production relative to wild-type. In certain embodiments, the disclosure provides a modified bacteria with enhanced growth rate and methods of producing modified cyanobacteria capable of enhanced growth rate relative to wild-type. In particular, Applicants have demonstrated that the ability of e.g., Synechococcus elongatus PCC 7942, to adapt to high salt concentrations introduced in the environment can be used to drive adaptive evolutionary changes providing strains with desirable traits.

All references to singular characteristics or limitations of the present disclosure shall include the corresponding plural characteristic or limitation, and vice versa, unless otherwise specified or clearly implied to the contrary by the context in which the reference is made.

The following examples illustrate features and/or advantages of the compositions, systems, and methods according to the general inventive concepts. The examples are given solely for the purpose of illustration and are not to be construed as limitations of the general inventive concepts, as many variations thereof are possible without departing from the spirit and scope of the general inventive concepts.

### EXAMPLES

### Growth Conditions

All evolved strains used in this study were maintained and transferred monthly on BG11(NO₃⁻) agar plates supplemented with 5 μg/mL of kanamycin in a continuous light chamber set at approximately 28-30 μmol s⁻¹ m⁻² and 29-32° C. Evolved strains transformed with the sucrose permease gene (cscB) are maintained on BG11(NO₃⁻) agar plates supplemented with 5 μg/mL of kanamycin and chloramphenicol and strains obtained from Dr. Ducat at Michigan State University including the CscB (Ducat) strain were maintained on BG11 agar plates supplemented with 5 μg/mL of chloramphenicol at the same conditions.

### Accelerated Evolution and Screening of Sucrose-Producing Strains

Flasks of mutS0: nirAp (Km^{R}) were grown in BG11(NO₃⁻) until the OD at 730 nm was approximately 0.7-1.0. The culture was centrifuged and the supernatant was removed. The pellet was washed three times using BG11(NH₄⁺) medium and then resuspended. In a 6 well plate, BG11(NH₄⁺), resuspended cells, kanamycin (5 μg/mL), and NaCl prepared in BG11(NH₄⁺) and filter sterilized at a final concentration of 150 mM was added. The culture was incubated on a shaker at 30 μmol s-1 m⁻² and 30° C. for 4 days. Following the 4 days, 0.5 mL of cells were removed and centrifuged. The pellet was washed with BG11(NO₃⁻), serially diluted, and 100 μL of each dilution was spread plated on BG11(NO₃⁻)+kanamycin agar plates. The plates were incubated in a continuous light chamber until individual isolated colonies appeared which were transferred to a new BG11(NO₃⁻)+kanamycin agar plate for maintenance.

The second round of evolution was performed on Se1-1086 in the same manner as the primary round however, the final concentration of NaCl used for this round of evolution was 200 mM.

The third round of evolution was performed on mSe2-842 in the same manner as the primary round. However, the final concentration of NaCl used for this round of evolution was 250 mM.

### Manual Sucrose Assay

All reagents for the sucrose assays were obtained and prepared following the protocols provided in the sucrose/D-glucose assay kits (Megazyme). Colonies to be used for sucrose assays were transferred from agar plates to liquid medium containing 96 well plates. The cells were incubated on a shaker for 40-48 hours at 30° C. and 50 μmol s⁻¹ m⁻² continuous light prior to salt induction for the primary and secondary screening. For the third round of screening, cells were incubated on a shaker for 24 hours at 37° C. and 50 μmol s⁻¹ m⁻² continuous light prior to salt induction. Salt induction was carried out using 25 μL of a stock solution of NaCl, prepared in liquid BG11 (NO₃⁻) then filter sterilized, and the 96 well plates were incubated as previously described. Following the induction period, the optical density of the cultures was measured at 730 nm using the SpectraMax® iD5 microplate reader (Molecular Devices) and sucrose assays were performed. In the assay plates, 50 μL lysis solution (1% DTAB in 0.2M NaOH) was mixed with 25 μL of the induced culture. Following lysis of the cells, 25 μL of 0.4M HCl was added to neutralize the pH. To one set of technical replicates, 50 μL of sodium acetate buffer was added and to another set of technical replicates, 50 μL of a premixed invertase solution (45 μL of sodium acetate and 5 μL of 8 U/mL invertase) was added. The assay plate was incubated for 45 minutes at 50° C. in the dark. Following this initial incubation 100 μL of GOPOD reagent was added to each well, incubated at 50ºC for 20 minutes and the absorbance was read at 510 nm.

### High-throughput Sucrose Assays

High-throughput sucrose assays were performed in the epMotion 5073 liquid dispenser (Eppendorf) using the same sucrose assay method as previously described with some minor changes. For the high-throughput assays, 50 μL of culture was used followed by the addition of 25 μL of the lysis buffer (2% DTAB in 0.4 M NaOH) which was mixed continuously for 10 minutes. All remaining steps of this assay were consistent with those previously described.

### Quantification of Sucrose

Sucrose standards of 0.002-0.1 mg/ml were used for sucrose assays following the same protocol as the samples described above. The averages for the technical replicates of the wells with and without the invertase solution were calculated. Using these two averages the absorbance of the invertase solution containing wells could be normalized using the averages of those that did not. A standard curve was constructed by plotting the normalized absorbance for each standard versus the concentration of sucrose in the standard. The linear equation of this curve was used to calculate the unknown sucrose concentration of the samples in the assay plates by using the normalized absorbance of each sample calculated in the same manner above. Any negative normalized absorbance values were set at 0 prior to being used in the standard curve.

### Generation and Confirmation of the Mutator Strain

Real-Time PCR was used to examine the expression of mutS following exposure of the cells to media containing ammonium as the nitrogen source. After just 30 minutes of exposure to NH₄⁺ containing medium the expression of mutS was notably reduced compared to NO₃⁻medium. As incubation of the cells were continued the expression of mutS was steadily decreased in the ammonium containing BG11. Spot assays were implored as an additional method to confirm decreased expression of the mutS gene in the NH₄⁺ containing medium through decreased growth of cells on agar plates containing either NH₄+ or NO₃⁻ as the only nitrogen source. Cells grown on media containing nitrate as the sole nitrogen source showed normal growth in all dilutions, whereas cells grown on media containing ammonium failed to grow in all dilutions except for the original concentration indicating a block in mutS expression leading to mutations and an inhibition of growth.

### Extracellular Sucrose Production

To examine extracellular sucrose productivity of evolved strains, a plasmid was obtained from Dr. Ducat at MSU in which the sucrose permease gene (cscB) from Escherichia coli was cloned into a neutral site 3 vector under the control of an Isopropyl β-D-1-thiogalactopyranoside (IPTG)-inducible promoter. Strains that were to be transformed were grown in a flask to the OD₇₃₀ nm of approximately 0.8-1. Once dense the strains were transformed overnight with ˜ 210 ng of the construct at 30° C. in the dark. Transformants were selected on BG11 (NO₃⁻) agar plates supplemented with 5 μg/mL of kanamycin and chloramphenicol respectively.

### Primary Screen of Evolved Strains Show Potential Elite Sucrose Producers:

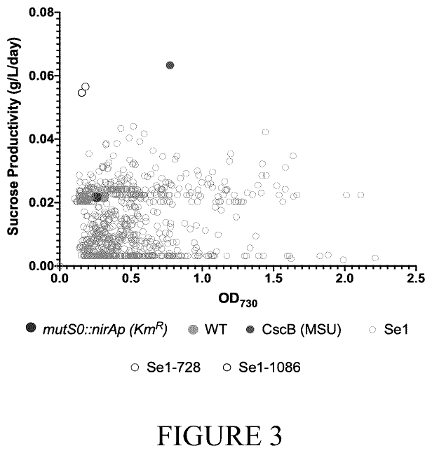

Over 700 evolved strains of PCC 7942 (Se1) during the first round of screening were produced (in BG11 (NH₄⁺) and 150 mM NaCl) and screened for their sucrose producing abilities. Following 18 hours of induction at 125 mM NaCl the wild-type (mutS0:nirAp (Km^{R})) exhibited approximately 0.02 g/L/day intracellular sucrose being produced. Se1 strains exhibited a large diversity in intracellular sucrose productivity compared to the wild-type strains (FIG. 3).

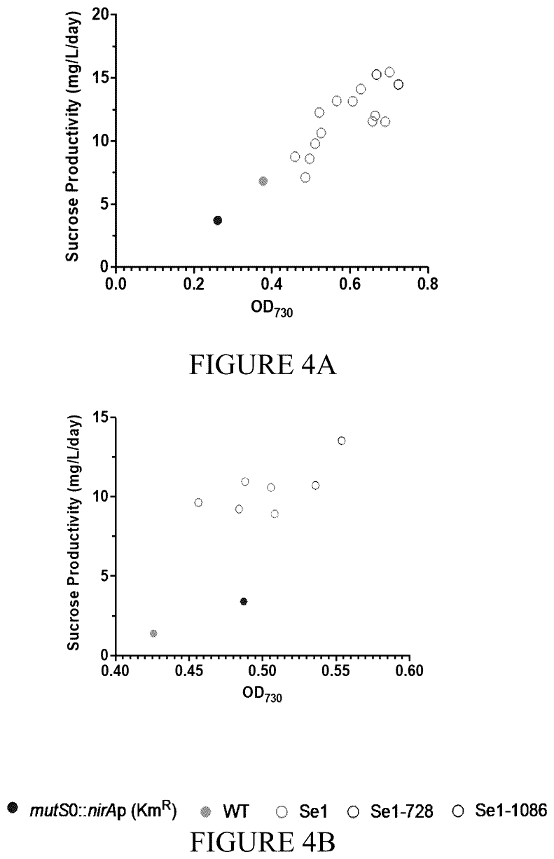

Analyzing intracellular sucrose can lead to a greater variability in the amount of sucrose produced because of the dependence on the metabolic state of individual cells making it difficult to get reproducible and accurate readings. To account for this variability and confirm increased sucrose production in the evolved strains, Se1 strains with a 2-fold or greater increase in sucrose productivity compared to WT were selected for confirmation analysis. Confirmation of intracellular sucrose productivity was completed in 150 mM NaCl to gain a better understanding of productivity in the conditions the strains were evolved in. WT and mutS0:nirAp strains demonstrated similarities in their intracellular sucrose productivity following an 18-hour induction in 150 mM NaCl as previously displayed. Se1 strains that had previously exhibited a 2-fold increase in sucrose productivity compared to the WT in the primary screen had more diversity in their sucrose producing capabilities (FIG. 4A). Another confirmation assay was performed on the strains exhibiting a 3-fold increase in intracellular sucrose productivities compared to the WT. All these selected Se1 strains demonstrated increased sucrose productivities in the second confirmation analysis. However, strains Se1-728 and Se1-1086 were still among the highest producers exhibiting a more than 2-fold increase in intracellular sucrose productivity compared to WT in both confirmation assays (FIG. 4B).

Confirmation of High Producing Strains from Primary Screen by Engineering a Sucrose Permease

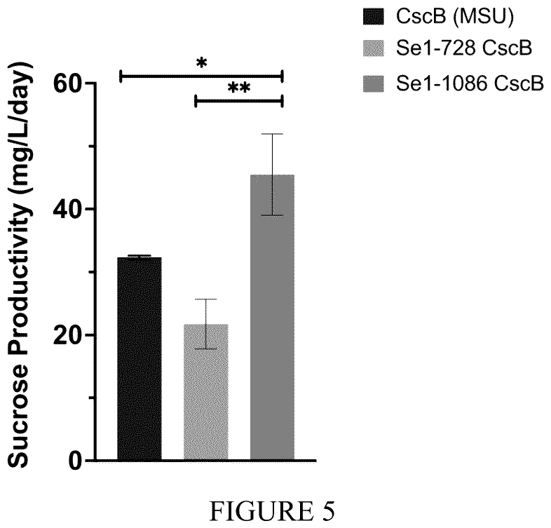

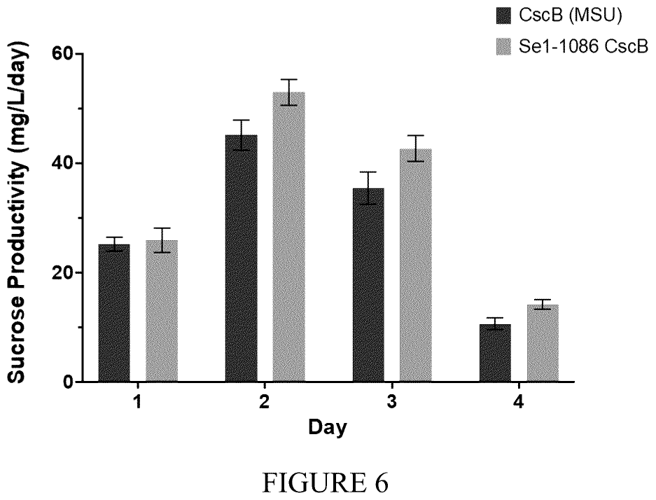

With mSe1-728 and mSe1-1086 showing the highest increase in intracellular sucrose productivity compared to the other mSe1 strains, they were transformed with the cscB plasmid. To determine which strain had greater extracellular sucrose productivity the strains were induced with 150 mM NaCl in 96-well plates and overexpression of cscB was induced with 1 mM IPTG (FIG. 5). mmSe1-1086 CscB exhibited significantly higher extracellular sucrose productivity after 18 hours with 45.51 mg/L/day compared to both mSe1-728 CscB and CscB (MSU), which were only able to export 21.73 and 32.34 mg/L/day, respectively. To gauge the larger scale extracellular sucrose producing capability of Se1-1086 CscB compared to CscB (MSU) both were grown in a multi-cultivator in 150 mM NaCl and 1 mM IPTG at an OD of 0.3 for 4 days (FIG. 6). After 24 hours in the salt, both strains showed relatively similar sucrose productivities of 25 mg/L/day. On the second day of salt induction mSe1-1086 CscB exported 52 mg/L/day of sucrose surpassing the potential of CscB (MSU) which only exported 45 mg/L/day however, when statistically analyzed was determined to be non-significant. Both strains showed a similar rate of decline in extracellular sucrose productivities following the third day of salt induction indicating detrimental strain on the cells. Following a single round of evolution, mSe1 -1086 showed an increase in both intracellular and extracellular sucrose productivity however, the overall increase exhibited was less than 2-fold and was determined to not be statistically significant. Overall, two strains, Se1-728 and Se1-1086, showed a 2.5-fold increase in sucrose productivity compared to the wild-type strains in the first round of evolution and screening experiment with 150 mM NaCl as the stressor.

### Growth Analysis of Evolved Fast-Growers

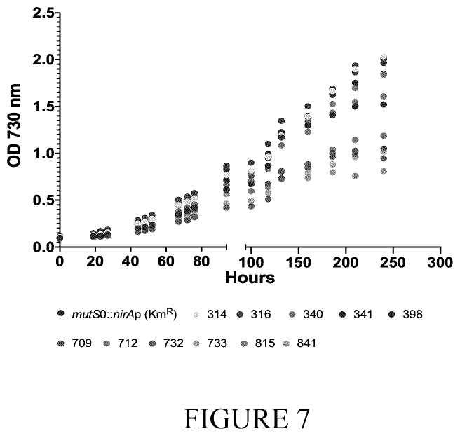

Faster-growing Se1 strains were selected for growth curves in a 24 well plate over a 10-day period. Prior to induction with salt, the Se1 strains show similar growth compared to the wild-type. After 4 days the cells were induced with 125 mM NaCl and WT cells showed continuous growth at a steady rate following this induction. Some of the Se1 strains showed an inability to continue growth following salt induction. However, a few strains, Se1-314, Se1-316, and Se1-340 show increased growth following salt induction indicating some low concentration salt tolerance on a small-scale level compared to wild-type (FIG. 7).

Second Round of Accelerated Evolution on mSe1-1086 Shows Further Enhancement of Sucrose Productivity

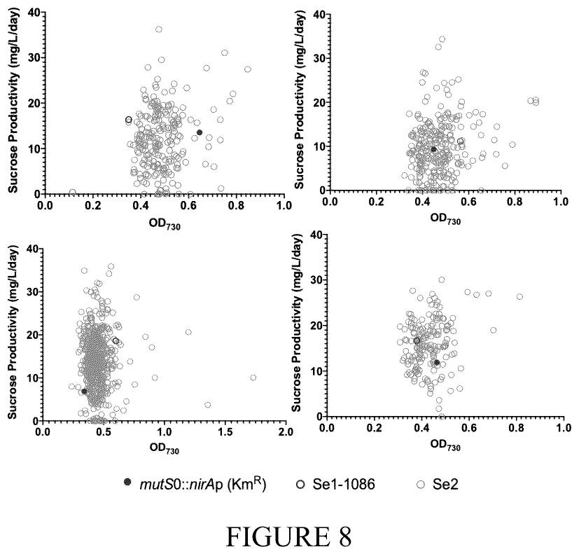

With the increased sucrose productivity exhibited by the mSe1-1086 strain, it was determined to be the highest sucrose-producing mutant from the first round of evolution. In an attempt to further improve sucrose productivity, the Se1-1086 strain was used for a second round of evolution in 200 mM NaCl. Following this second round of evolution, approximately 1200 evolved strains (mSe2) were produced and screened for their intracellular sucrose producing capabilities. Similar to the first round of evolution, mSe2 strains showed diverse intracellular sucrose productivity compared to mutS0:: nirAp and mSe1-1086 after 30 hours of induction in 150 mM NaCl (FIG. 8). The majority of the mSe2 strains had either no change or a reduction in their sucrose productivity compared to Se1-1086. However, there were more mSe2 strains that exhibited a 2-fold or higher sucrose productivity compared to mutS0:: nirAp and Se1-1086 than the first round of evolution. With the production of sucrose being heavily reliant on the cellular conditions such as availability of intermediate pools, confirmation assays were performed in biological replicates on strains that exhibited a 2-fold or greater increase in sucrose productivity compared to mutS0:: nirAp.

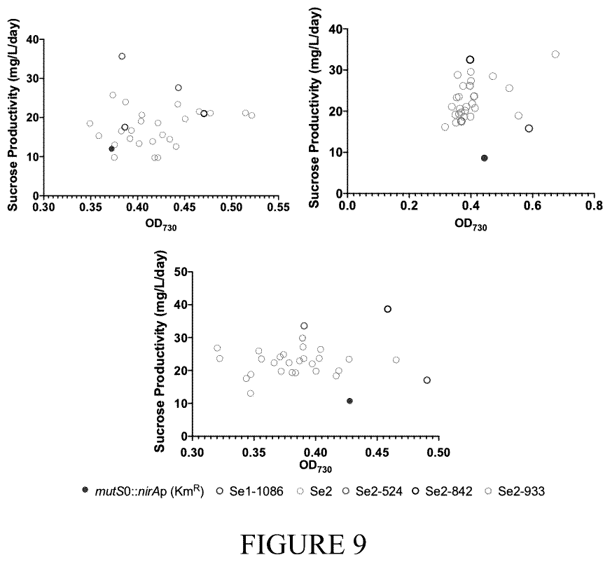

Three confirmation assays were performed to identify any potential elite sucrose producers from the second round of evolution for scale-up analysis. Between the three confirmation assays, a large portion of the mSe2 strains had discrepancies in their sucrose productivity after 30 hours in 150 mM NaCl. However, when examining the sucrose productivity of the mSe2 strains in each confirmation assay, the three strains that demonstrated the most consistent sucrose productivity were mSe2-524, 842, and 933 with average productivities of 26.1, 30.7, and 30.5 mg/L/day, respectively. Between the confirmation assays, mSe2-524, mSe2-842, and mSe2-933 all exhibited more than a 1.5-fold increase in sucrose productivity compared to mSe1-1086 and a 2.5-fold increase compared to mutS0:nirAp (FIG. 9). The consistency and high sucrose-producing capabilities of mSe2-524, mSe2-842, and mSe2-933 made them of interest to examine extracellular sucrose production. They were transformed with the cscB plasmid to examine their sucrose exporting capacity in scale-up analysis.

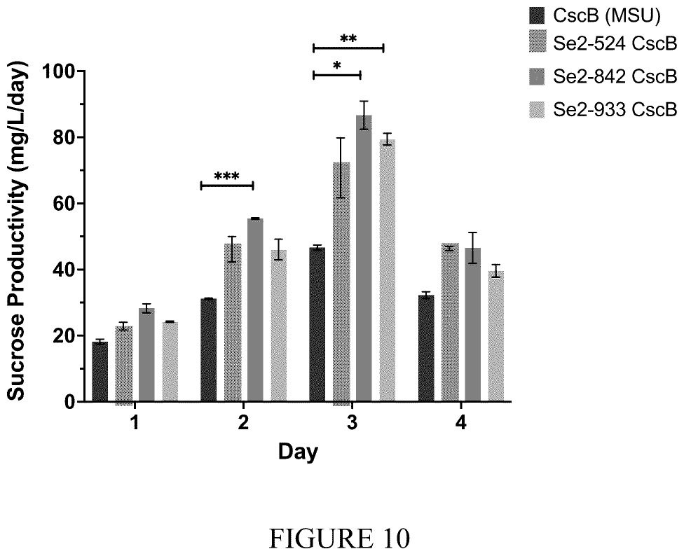

Upon successful transformation of the mSe2 strains with the cscB plasmid, transformants were scaled up in the multi-cultivator with 150 mM NaCl and 1 mM IPTG for 4 days. Following 24 hours of salt stress, the three mSe2 CscB strains all revealed a slightly increased extracellular sucrose productivity compared to CscB (MSU) (FIG. 10). When sucrose productivity was examined following 48 hours strains Se2-524 CscB and mSe2-933 CscB both exhibited a 1.5-fold increase in extracellular sucrose compared to the CscB (MSU) strain where mSe2-842 CscB demonstrated a 1.8-fold increase. The highest level of extracellular sucrose productivity was observed on the third day of salt induction in all strains. mSe2-524 CscB and mSe2-933 CscB displayed a 1.5- to 1.7-fold increase in extracellular sucrose productivities where mSe2-842 had a 1.9-fold increase compared to the CscB (MSU) strain. All strains showed a sharp decline in sucrose productivity following the fourth day in salt, likely due to a decrease in the density of the culture indicating cell death had begun to occur. These results revealed that following the second round of evolution, strains were isolated that illustrated an increased capacity to produce sucrose compared to CscB (MSU). In addition to this, these same strains also demonstrated the ability to produce sucrose over a longer period of time.

Third-Round High Throughput Screening with More Mutants with Beneficial Phenotypes

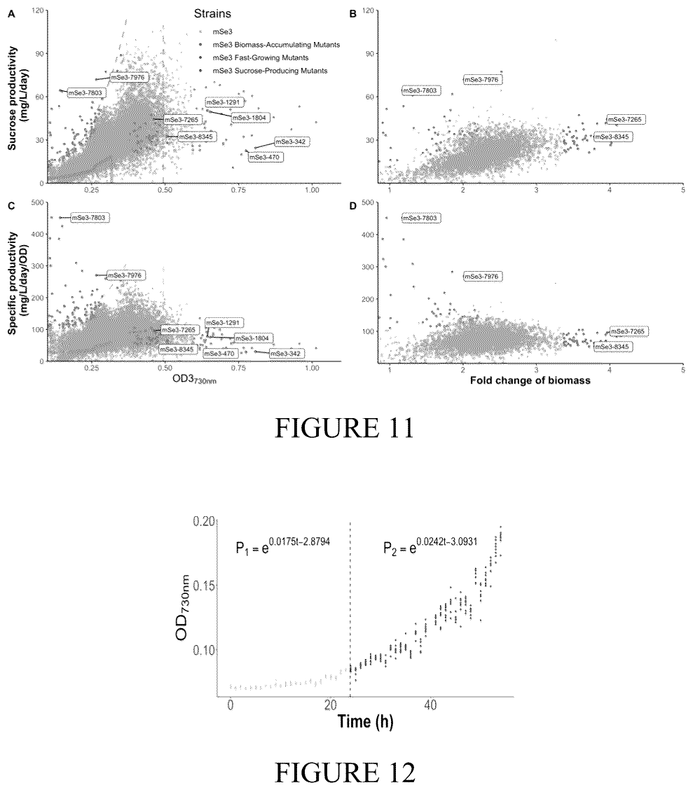

From the third round onward, a more comprehensive strategy was adopted, searching for Sucrose Producing Mutants (SPMs), Biomass-Accumulating Mutants (BAMs), and Fast-Growing Mutants (FGMs). An improved photosynthetic system was considered to enhance carbon flux, reflected in growth rate, biomass accumulation and sucrose productivity. A linear model was developed using a training dataset tested on mSe0, and mutants with low p-values were considered candidate strains for SPM, BAM, or FGM. FGM screening utilized fold change as a proxy for biomass accumulation, while BAM screening considered terminal OD730 nm. The mutants with the lowest p-values were isolated for subsequent validation. EpMotion m5073 was applied for liquid handling, SpectroMax iD5 by Molecular Device was used for speed reading of optical density (FIGS. 11 and 12).

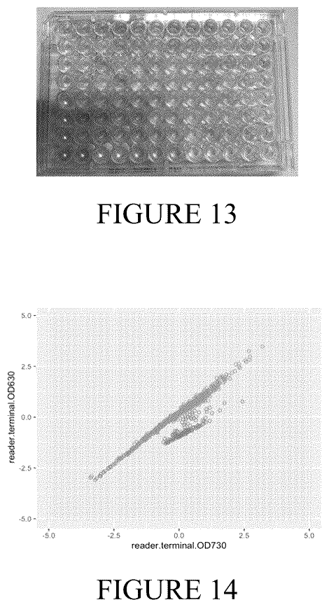

During this round of screening, chlorosis of cyanobacteria was identified as the yellow appearance of bacterial culture, which indicated the lower viability of cyanobacterial mutants in the stress condition. Thus, the identification of chlorosis can be used as another proxy to assess the robustness of cyanobacteria under stress condition. In order to classify the chlorosis status, a simple neural network was used to differentiate mutants that did not undergo chlorosis from those that did. In comparison, a logistic classifier was also built with 66.9281% of accuracy, while our neural network model achieved 95.42484% of accuracy (FIGS. 13 and 14).

Validating Beneficial Phenotypes under Salinity Stress

To overcome noise in the linear model training, candidate strains were validated with ample replicates. The top 1% of mSe3 sucrose-producing mutants and the top 0.5% biomass-accumulating mutants were selected for the validation assay. In order to achieve sufficient power, a power analysis was conducted for both group mutants. Block ANOVA was further conducted to identify potential candidate strains.

For an effect size of 0.1 difference of population biomass, 12.1 samples are needed to achieve 90% power; in the validation assay, 12 (4 replicates X 3 batches) replicates have the power of 89.7% to detect a difference of 0.1 of population biomass.

Using the biomass validation data, 8 mutants were identified as candidate strains by block ANOVA, including mSe3-342, 358, 470, 1291, 1804, 7265, 8345, and UTEX 2973 (control strain). Among these, mSe3-358 had significantly lower biomass compared to the control PCC 7942 strain.

The 95th percentile of the sucrose productivity residuals for the mSe3 mutants was about 21, whereas the average residual of the controls was about −4. We thus used an effect size of 25 residual sucrose productivity for the power analysis. To reach 90% power, 11 samples per group are needed. In the validation assay, 16 replicates (8 replicates X 2 batches) have the power of 98% to detect a difference of 25 in sucrose productivity. One concern we had was the skewness of log-transformed sucrose production control data used for the power analysis; however, the validation data showed that adjusted log sucrose productivity is not very skewed.

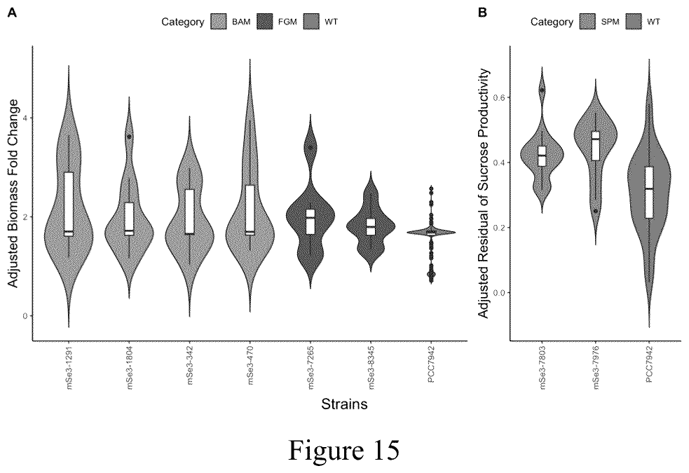

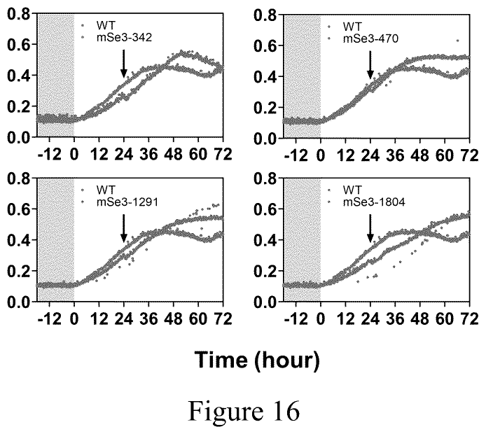

Only 2 sucrose-producing mutants mSe3-7803 and mSe3-7976 were identified as the candidate strains by block ANOVA. There were 3 others (mSe3-5892, 8820, and 8828) that had significantly lower sucrose production. Power analysis suggested that 24 replicates were sufficient to discern true differences. All candidate mutant strains were inoculated as octuplicates, and metrics were calculated from validation data to verify beneficial phenotypes. BAM strains mSe3-342, mSe3-470, mSe3-1291, mSe3-1804, FGM strains mSe3-7265, and mSe3-8345, and SPM strains mSe3-7803 and mSe3-7976 demonstrated differentiable beneficial phenotypes under high-salinity stress conditions (FIG. 15). While the biomass accumulation phenotype was confirmed for all BAM strains, the fast-growing phenotype in FGM strains was not reflected in growth dynamics, suggesting that mutations in FGM strains may contribute to viability under salinity stress (FIG. 16).

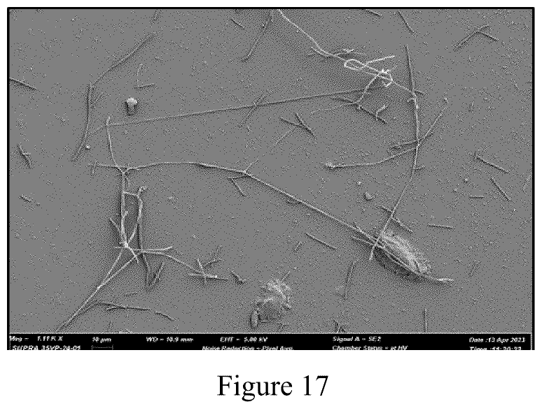

Several candidate mutants with beneficial phenotypes exhibited a filamentous shape. Notably, mSe3-342 displayed the most distinguishable length compared to wild-type strains (FIG. 17), raising questions about whether the filamentous shape aids biomass accumulation.

All combinations of method or process steps as used herein can be performed in any order, unless otherwise specified or clearly implied to the contrary by the context in which the referenced combination is made.

All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all subranges between (and inclusive of) the minimum value of 1 and the maximum value of 10; that is, all subranges beginning with a minimum value of 1 or more (e.g., 1 to 6.1), and ending with a maximum value of 10 or less (e.g., 2.3 to 9.4, 3 to 8, 4 to 7), and finally to each number 1, 2, 3, 4, 5, 6, 7, 8, 9, and 10 contained within the range.

The compositions, systems, and corresponding methods of the present disclosure can comprise, consist of, or consist essentially of the essential elements and limitations of the disclosure as described herein, as well as any additional or optional ingredients, components, or limitations described herein or otherwise useful in the general inventive concepts.

The compositions of the present disclosure may also be substantially free of any optional or selected component or feature described herein, provided that the remaining composition still contains all of the required elements or features as described herein. In this context, and unless otherwise specified, the term “substantially free” means that the selected composition contains less than a functional amount of the optional component, typically less than 0.1% by weight, and also including zero percent by weight of such optional or selected component.

To the extent that the terms “include,” “includes,” or “including” are used in the specification or the claims, they are intended to be inclusive in a manner similar to the term “comprising” as that term is interpreted when employed as a transitional word in a claim. Furthermore, to the extent that the term “or” is employed (e.g., A or B), it is intended to mean “A or B or both A and B.” When the Applicant intends to indicate “only A or B but not both,” then the term “only A or B but not both” will be employed. Thus, use of the term “or” herein is the inclusive, and not the exclusive use. In the present disclosure, the words “a” or “an” are to be taken to include both the singular and the plural. Conversely, any reference to plural items shall, where appropriate, include the singular.

In some aspects, it may be possible to utilize the various inventive concepts in combination with one another. Additionally, any particular element recited as relating to a particularly disclosed embodiment should be interpreted as available for use with all disclosed embodiments, unless incorporation of the particular element would be contradictory to the express terms of the embodiment. Additional advantages and modifications will be readily apparent to those skilled in the art. Therefore, the disclosure, in its broader aspects, is not limited to the specific details presented therein, the representative apparatus, or the illustrative examples shown and described. Accordingly, departures may be made from such details without departing from the spirit or scope of the general inventive concepts.

While the invention has been illustrated and described in detail in the drawings and foregoing description, the same is to be considered as illustrative and not restrictive in character. It should be understood that only the exemplary embodiments have been shown and described and that all changes and modifications that come within the spirit of the invention are desired to be protected.

1. A method of identifying a genetic mutation capable of enhancing at least one of photosynthetic rate and photosynthetic efficiency, the method comprising:
modifying of a plurality of bacterial cells to form modified host cells to enhance mutation rate relative to wild-type organisms;
culturing the modified bacterial cells in the presence of an environmental stressor;
identifying modified cells that demonstrate a marker of enhanced photosynthetic rate and/or efficiency; and
selecting the identified modified cells showing enhanced photosynthetic rate and/or efficiency.

2. The method of claim 1, wherein the environmental stressor comprises a medium designed to increase a rate of adaptive mutation.

3. The method of claim 2, wherein the medium comprises an increased concentration of a salt in the range of 100 mM to about 500 mM.

4. The method of claim 3, wherein the salt is sodium chloride.

5. The method of claim 1, wherein modifying of a plurality of bacterial cells to enhance mutation rate relative to wild-type organisms comprises introducing a nirA promoter via a vector to form a modified cell.

6. The method of claim 5, wherein the promoter is introduced in front of a mismatch repair pathway gene.

7. The method of claim 1, wherein the bacterial cells are cyanobacterial cells.

8. The method of claim 1, wherein the bacterial cells are Synechococcus elongatus PCC 7942.

9. The method of claim 1, wherein identifying the modified cells comprises a high throughput screening for sucrose production based on modeling.

10. The method of claim 1, wherein identifying the modified cells comprises a high throughput screening for cells displaying enhanced biomass accumulation as measured by optical density.

11. A method of achieving greater photosynthetic rate and/or efficiency in a bacterial species, the method comprising:
modifying of a plurality of bacterial cells to enhance mutation rate relative to wild-type organisms;
culturing the modified bacterial cells in the presence of an environmental stressor;
identifying modified cells that demonstrate an enhanced marker of photosynthetic rate and/or efficiency;
identifying the modified cells that produce the enhanced photosynthetic rate and/or efficiency;
isolating the genetic information for the genetic modification; and
introducing a vector comprising the genetic modification into a plurality of second host cells.

12. The method of claim 11, wherein the environmental stressor comprises a medium designed to increase a rate of adaptive mutation.

13. The method of claim 12, wherein the medium comprises an increased concentration of a salt.

14. The method of claim 13, wherein the salt is sodium chloride.

15. The method of claim 11, wherein the bacterial cells are cyanobacterial cells.

16. The method of claim 11, wherein the bacterial cells are Synechococcus elongatus PCC 7942.

17. The method of claim 11, wherein the genetic modification provides at least one of enhanced sucrose production, enhanced biomass accumulation, and enhanced growth rate.

18. A method of stimulating adaptive mutation in a host cell to produce phenotypic changes of interest, the method comprising:
modifying of a plurality of cyanobacterial cells to form modified cyanobacterial cells;
culturing the modified cyanobacterial cells in the presence of an environmental stressor;
identifying modified cells that demonstrate at least one phenotypic change of interest; and
further culturing the identified cells in the presence of an environmental stressor.

19. The method of claim 18, wherein the environmental stressor comprises an increased concentration of sodium chloride.

20. The method of claim 19, wherein modifying of a plurality of bacterial cells comprises introducing a nirA promoter, wherein the promoter is introduced in front of a mismatch repair pathway gene, and wherein the phenotypic change of interest is selected from the group comprising enhanced sucrose production, enhanced biomass accumulation, and enhanced growth rate.
